(19) United States
(12) Patent Application Publication
(10) Pub. No.: US 2018/0008448 A1
(43) Pub. Date: Jan. 11, 2018

(54) BANDAGE

(71) Applicant: OTTO BOCK HEALTHCARE GMBH, Duderstadt (DE)

(72) Inventor: Matthias VOLLBRECHT, Herzberg (DE)

(73) Assignee: OTTO BOCK HEALTHCARE GMBH, Duderstadt (DE)

(21) Appl. No.: 15/543,189

(22) PCT Filed: Dec. 18, 2015
(86) PCT No.: PCT/EP2015/080662

§ 371 Date: Jul. 12, 2017

(30) Foreign Application Priority Data
Jan. 14, 2015 (DE) 10 2015 000 441.7

(51) Int. Cl. A61F 5/01; A61F 5/03; A61F 5/02

(52) U.S. Cl. CPC A61F 5/0111; A61F 5/022; A61F 5/03

(57) ABSTRACT

A bandage having at least two textile segments which can be connected to each other, each textile segment comprising at least one loop made of a textile material and being arrangeable such that at least one connecting bar can be pushed through at least one loop of the one textile segment and through at least one loop (of the other textile segment.

The invention relates to a bandage having at least two textile segments which are interconnectable.

Bandages of this type have nowadays been known from the prior art for some time and are disclosed in WO 2007/003148, for example. Bandages of this type typically surround a body part of the wearer, said body part potentially being the torso, for example. The bandages typically have a supporting function and can be configured from elastic or non-elastic materials. In order for bandages of this type to be prevented from rolling up on the body of the wearer and for the support function to be simultaneously improved, it is known from the prior art for supporting elements, for example in the form of bars, to be incorporated into bandages of this type.

In particular in the case of torso bandages which are worn about the thorax of the wearer, it is expedient for said bandages to be designed to be adaptable to the body circumference of the respective wearer. It is therefore known from the prior art for bandages of this type to be made from a multiplicity of segments which can be interconnected and released from one another. In WO 2007/003148, this is achieved by jaw-type hook-and-pile fastener elements which are disposed at one end of the segments and encompass another end of another segment, the communicating hook-and-pile fastener elements being positioned on the external side of said other segment. In this way, an additional segment can be integrated in. the bandage in order for the bandage to be extended in length, for example. The individual segments can be interconnected for example by zip fasteners or metallic hinges, the individual parts of the latter being attached to the textile segments.

These solutions are described in WO 2007/018512 A1, example.

It is known from US 2008/0021357 A1 for torso bandages of this type to be surrounded. with. stabilizing straps in order for the stability required for the wearer of the bandage to be achieved. Said stabilizing straps are also composed of a multiplicity of segments which are attached to one another by way of hinges.

An orthopedic corset which is composed of elements that are connected in an articulated manner is known from DE 10 2012 017 645 B3. The individual elements herein on. one side dispose of a hinge element pin which can be introduced into a hinge element socket that is configured therefor on the neighboring segment. A screw is subsequently pushed through the two components thus interconnected an u is fixed by way of a nut.

It is disadvantageous in the solutions known, for example, that either a mutually relative positioning of the individual components that is as precise as possible has to be guaranteed in order for functioning of the bandage that is as optimal as possible to be achieved in the case of the use of jaw-type hook-and-pile fastener connections. For this purpose, however, interconnecting the individual components in the case of this design embodiment is possible in a particularly simple manner. By contrast, if other exemplary embodiments such as, for example, zip fasteners or the design embodiment described having a hinge socket and a hinge element are used, the mutual arrangement of the two components is possible in only one position since the two segments can only be interconnected in this position. However, the connection by way of a zip fastener, for example, or the screw connection already mentioned, is complex and occasionally not possible without tools.

Moreover, a zip fastener does not lead to sufficient stiffening of the bandage in the longitudinal direction such that rolling-up of one end or both ends cannot be prevented. Moreover, a zip fastener is cost-intensive.

The invention is therefore based on the object of proposing a bandage which disposes of interconnectable textile segments which are interconnectable in a simple and comfortable manner, at the same time are mutually positionable in only an optimal manner as far as possible, and nevertheless are producible in a simple and cost-effective manner.

The object set is achieved by the invention by way of a bandage having at least two textile segments which are interconnectable, wherein the bandage is characterized in that each of the two textile segments has one loop of a textile material and can be disposed on one another in such a manner that at least one connecting bar is fittable by sliding through at least one loop of the one textile segment and at least one loop of the other textile segment. A type of textile hinge is created when the connecting bar is fitted by sliding through the loops of the two textile segments to be connected. In an optimal manner, the loops of the two textile segments have lengths which collectively correspond to the length of the connecting bar or are somewhat longer than the length of the connecting bar. The loops are preferably disposed on the two textile segments such that said loops in the connected state bear on one another such that said loops completely surround the connecting bar such that the latter is not visible from the outside. An exact mutual positioning of the two textile segments is provided in this case since the two textile segments are interconnectable only in this state. This applies in particular to the case in which the two textile segments each have more than one loop. Moreover, a displacement of the textile segments in a direction parallel with the longitudinal direction of the connecting bar is precluded in this way.

A connecting bar in the context of the present invention is in particular understood to he anything that can be used for interconnecting two textile segments and at the same time has a sufficiently high flexural rigidity in order for the transverse and tensile forces that arise in the intended use of the bandage to be absorbed, on the one hand, and on the other hand to prevent that the textile segments are rolled up in the intended use of the bandage. If a bandage without the respective bars is placed about a body part, for example a torso, of a patient, there is the potential for the peripheries to roll up and to thus form a bead or a roll that encircles the respective body part. A connecting rod in the context of the present application has a sufficiently high flexural rigidity in order for said rolling up to be prevented. This is also referred to as erecting the textile segment. A connecting bar in the context of the present invention can consequently also be provided in the form of a wire, a rope, in particular a thick rope, or a cable element.

The connection by way of the “textile hinge” has a multiplicity of advantages. The individual textile segments can be interconnected and released from one another in a simple manner, even without a tool. Moreover, no metallic components are required, so that the production costs and the material costs are reduced. Moreover, the wear comfort of the bandage is enhanced since there is no longer the risk of metallic components potentially leading to unpleasant pressure points.

Should the loops have a certain degree of inherent strength and inherent stability, the two components to be interconnected can first be placed against one another or be positioned in the correct mutual position before the connecting bar in a further step is fitted by sliding through the individual loops. The connection is consequently a successful one even when the textile segments are mutually positioned in a manner that is clearly comparatively inaccurate, this not being the case with zip fastener connections that are known from the prior art, for example. The bandage wearer or an orthopedic technician can modify the length of the bandage in a simple and rapid manner by inserting or removing a textile segment, for example, without having to take any exaggerated care pertaining to the exact mutual positioning of the components. The connected segments subsequently are nevertheless mutually positioned in the desired manner.

The textile segments are advantageously composed at least largely of a textile material, wherein the at least one loop of the textile segment is also composed at least partially of the textile material. In one particularly advantageous design embodiment, the at least one loop is configured in an integral manner from that part of the textile segment that is composed of the textile material. In this way, the textile segment having the at least one loop can be produced in a particularly simple manner in that a respective element is cut from the textile material, for example, said respective element subsequently being able to be folded over and stitched such that the at least one loop results. The production method is simplified on account thereof, and the production costs are thus reduced.

Alternatively thereto, by folding over and stitching the textile material in the longitudinal direction of the bandage to be made, a long loop can be established across the entire length of the connection, those portions that are not required for the individual loop elements then being able to be released, for example cut or punched, from said long loop. On account thereof, a particularly accurate contour of the loop and of the intermediate spaces between two loops is achieved, this being particularly advantageous when establishing dove-tail shaped loops.

It has been demonstrated as advantageous for at least one loop of at least one textile segment to be reinforced with a reinforcement element which is preferably disposed on the inside of the loop. This here can be a further textile layer, for example, which in the production of the textile segment and/or in the production of the loop is already disposed on the loop material. On account thereof, the stability of the connection between the textile segments to be connected is increased. At the same time, the diameter of the loop can be reduced such that the connecting bar is more tightly surrounded by the respective loop. On account thereof, the mobility of the connection between the two textile segments can be controlled and set, for example. It can thus be expedient for same bandages that the ratio of the diameter of the at least one loop to the diameter of the connecting bar is chosen such that a pivotability of the two textile segments that are connected by way of the connecting bar is provided. However, at other locations or in other bandages it can be expedient for a connection between the two textile segments that is as stiff and as rigid as possible to be achieved. In this case, a reinforcement element can be disposed in the loop such that the cross section is reduced. Additionally or alternatively thereto, a reinforcement element by way of which the friction properties of the inside of the loop on the connecting bar can be influenced and reduced, for example, can also be used, for example. In this way, the pivotability of the two textile segments that are interconnected by way of the connecting bar can be reduced.

The reinforcement element is preferably composed of a plastics material and is preferably configured as a tubular or a tube-sock element. As has already been set forth, elements of this type can already be disposed in the production of the loop, for example by being folded over or folded back and subsequently being stitched in the loop.

In order to prevent that the connection element slides out of the loop when the connecting bar is being fitted by sliding into the at least one loop in which a reinforcement element is located, it has proven advantageous for the reinforcement element to be fastened into the loop, in particular to be adhesively bonded into the loop. However, it is disadvantageous herein that the reinforcement element can no longer be replaced and the pivotability of the connection between the two textile segments thus can no longer be set, for example.

In one preferred design embodiment, at least one, preferably both, of the textile elements disposes of at least two loops. On account thereof it is guaranteed that at least one loop of each individual textile segment is disposed between two loops of the respective other textile segment, on account of which an accurate and comparatively stable positioning of the individual textile segments is achieved. Moreover, the stress that acts on the connecting bar, for example when the bandage having the connected textile segments is under tensile stress, is distributed across a plurality of points such that the stability of the bandage in particular in terms of tensile stress is reinforced. Moreover, it is possible for the individual loops of the textile segments to be made such that the end faces of the loops contact the end faces of the loops of the respective other textile segment when the two textile segments are positioned in the position in which they are intended to be connected. On account thereof, a retention force that prevents slippage or displacement of an individual textile segment relative to the respective other textile segment is initiated even when no connecting bar has yet been introduced into the loops.

in one preferred design embodiment, at least one loop of at least one textile segment is closable by at least one closure element. This is preferably the loop that is disposed at the bottom when the bandage is worn. If this loop is closed by a closure element, the connecting bar cannot slip out in a downward manner, a durable connection thus being guaranteed. A further loop is preferably closable by way of a closure element, the former being the uppermost loop. In this way, the connecting bar cannot slip out of the loop arrangement at any of the two sides, thus precluding any inadvertent release of the connection between the two textile segments.

The closure element is advantageously a foldable flap which in the closed position is fastenable preferably by a hook-and-pile fastener. A flap of this type is readily producible, does not lead to any unpleasant pressure points or to any other wear discomfort, and can be readily opened and closed without a tool.

At least one loop of each of the two textile segments to be connected is advantageously closable by way of a closure element. The closure elements can be of identical or dissimilar configuration. Alternatively or additionally to the foldable flap already described, the closure element can also be provided in the form of a hook-and-pile fastener or as a snap fastener, for example, or be configured as an additional bar.

The connecting bar advantageously disposes of a non-circular, preferably an oval or a rectangular, cross section. The pivotability of the two textile segments that are connected by way of the connecting bar can be set by the choice of the cross section. For example, if the pivotability is intended to be provided across a wide angular range, a connecting bar having a circular cross section has been proven advantageous. A connecting bar having a non-circular cross section is of advantage for a rigid connection between the two textile segments.

In one preferred design embodiment, the bar on at least one end disposes of a hook or engagement element by way of which said bar can engage in cavities, depressions, or introduction openings, provided for this purpose in at least one of the textile segments to be interconnected, if said bar has been brought to bear sufficiently far into the respective loops. It is thus conceivable, for example, for a tunnel that is continuous or else open only toward one side to be provided on at least one of the two textile segments, a respective hook element that is disposed on one end of the connecting bar being able to engage in the introduction opening of said tunnel. It is in this way prevented that the connecting bar can be completely fitted by sliding through the loops or in the use of the bandage by virtue of the mechanical stresses or movements arising therein can slip out of the loops in a downward manner, on the one hand. A rotation of the Connecting bar is prevented, on the other hand. In one particularly preferred design embodiment, a connecting bar at one end disposes of two hook elements which can be disposed on two opposite sides. In this way, the bar can engage in each case in one of the tunnels that is open or closed on one side of one of each of the textile segments to be connected.

Alternatively or additionally thereto a fastening element can also be fastened to the connecting bar and/or integrated into the connecting bar. This fastening element can be an element of a snap fastener or of a hook-and-pile fastener element, for example, while the respective other element is positioned on a loop of one of the textile segments to be connected, for example. Once the connecting bar has been fitted by sliding into the respective loops the snap fastener or hook-and-pile fastener element on the connecting bar can thus be brought to engage with and contact the corresponding counter element on the loop such that the connecting bar is fixed to the respective loop and thus to the textile segment. Slipping out is likewise positively avoided in this way.

The respective element of the snap fastener or hook-and-pile fastener element or of other locking or fastening elements can be adhesively bonded to the connecting bar or be fastened thereto in another way.

A bandage according to the invention advantageously disposes of more than two, for example four or six, textile segments which particularly advantageously each dispose of loops such that the textile segments can be interconnected in pairs by in each case one or a plurality of connecting bars. On account thereof, a bandage that is capable of being employed in a very flexible manner is achieved, since textile segments can be installed and removed from the bandage in a very simple yet stable manner in. order for the length of the bandage to be set to the body circumference or to the circumference of a body part of the wearer of the bandage, for example.

The at least one to on the respective textile segment can have various shapes. Straight loops and dove-tail shaped or triangular loops are equally conceivable The two last-mentioned embodiments in particular prevent or at least impede the textile segments from being able to be pulled apart from one another in the circumferential direction of the bandage, even when no connecting bar has been routed through the loops. Alternatively or additionally thereto it is possible for the loops of at least one of the two textile segments to be interconnected, preferably of both textile segments to be interconnected, to be configured such that said loops can be opened. In this way, a connecting bar that is located in the loops can be removed in a particularly simple manner, for example, and/or a connecting bar which is connected fixedly and unreleasably to at least one of the textile segments can be used. The at least one loop of at least one of the textile segments is in this case present in an open state. Said loops herein can be shaped such that the one end thereof comes to lie on that other end that points toward the main part of the textile segment. If mutually communicating elements of a connecting or locking element, for example mutually communicating elements of a snap fastener or of a hook-and-pile fastener element, axe disposed on the two faces that in this instance bear on one another, the loop can be closed in a simple manner and be reopened accordingly in this way.

In one further embodiment of the present invention, in each case one connecting bar is introduced into the respective loops from both sides. This can be performed from above and from below, for example. The at least one connecting bar advantageously disposes of at least one fastening element and/or at least one hook element. The latter can advantageously be disposed on one end or on both ends of the connecting bar. If a plurality of connecting bars which can be introduced into the loops from different sides or are introduced sequentially from the same side are used, the different connecting bars are particularly advantageously interconnectable. This can be embodied as a plug connection, a hook-and-pile connection, or any another connection. In this way, it is possible for the at least one connecting bar to be fastened to at least one, but preferably to both, textile segments to be interconnected and for slipping out or displacement of the connecting bar in the use of the bandage to thus be avoided. If more than one connecting bar is used, said connecting bars are preferably fastened to one or both of the textile segments so as to be next to one another and/or advantageously in an individual or collective manner.

The interconnection between the connecting bars can also be established by way of a screw connection, for example. The connecting bars are advantageously shorter than the textile segments in the direction of the connection such that the former can be completely received in the loops of the two textile elements.

An exemplary embodiment of the present invention will be explained in more detail hereunder with the aid of the appended figures in which:

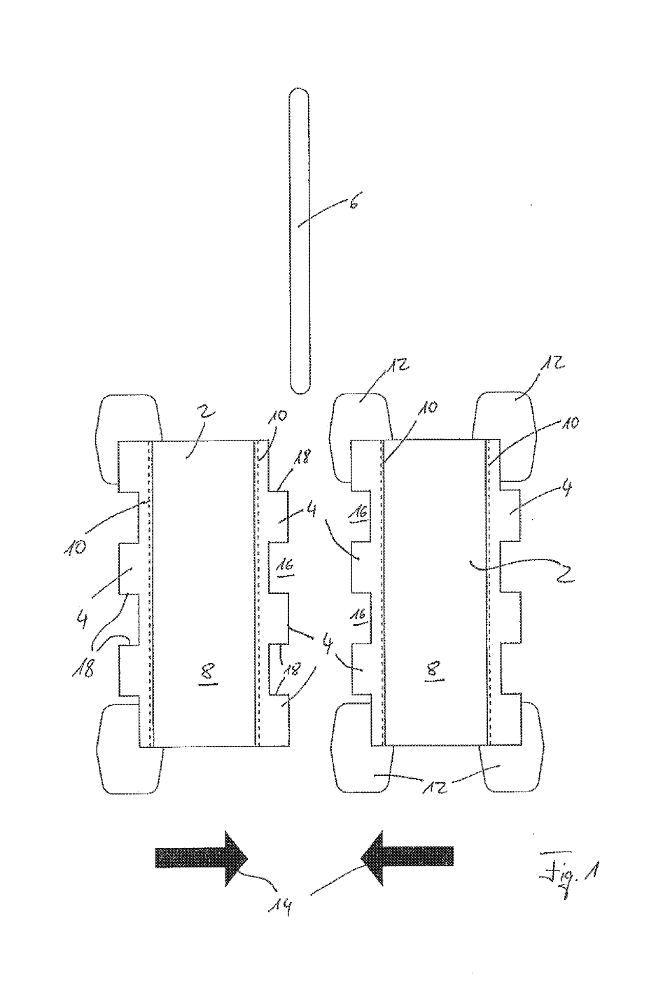

FIG. 1 shows two textile segments in the non-connected state;

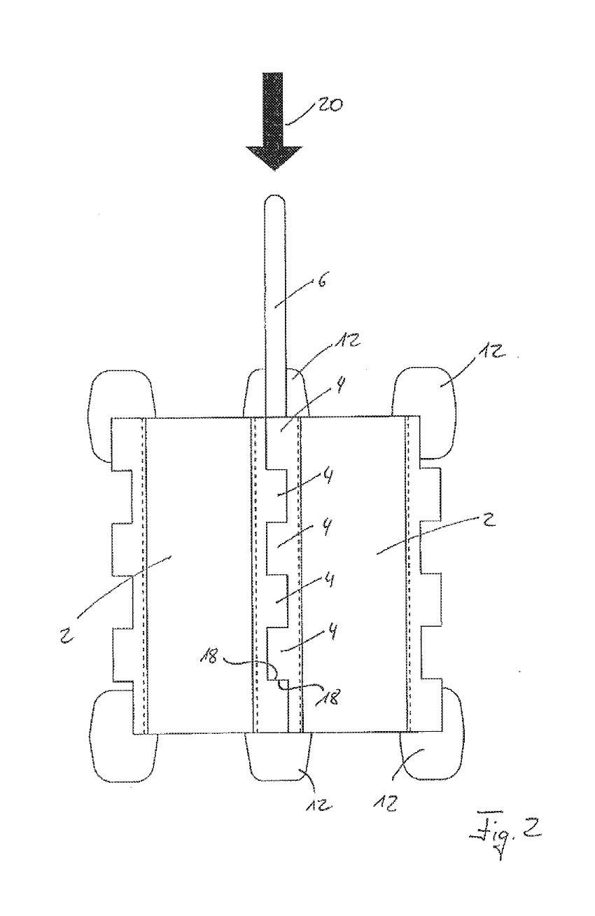

FIG. 2 shows two textile segments prior to the connecting bar being introduced;

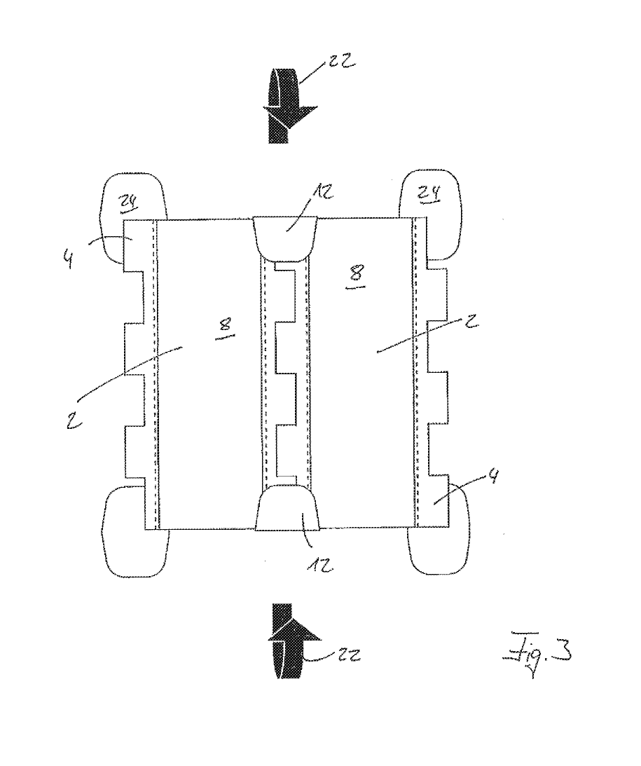

FIG. 3 shows two textile segments for a bandage according to one exemplary embodiment of the present invention, in the connected state;

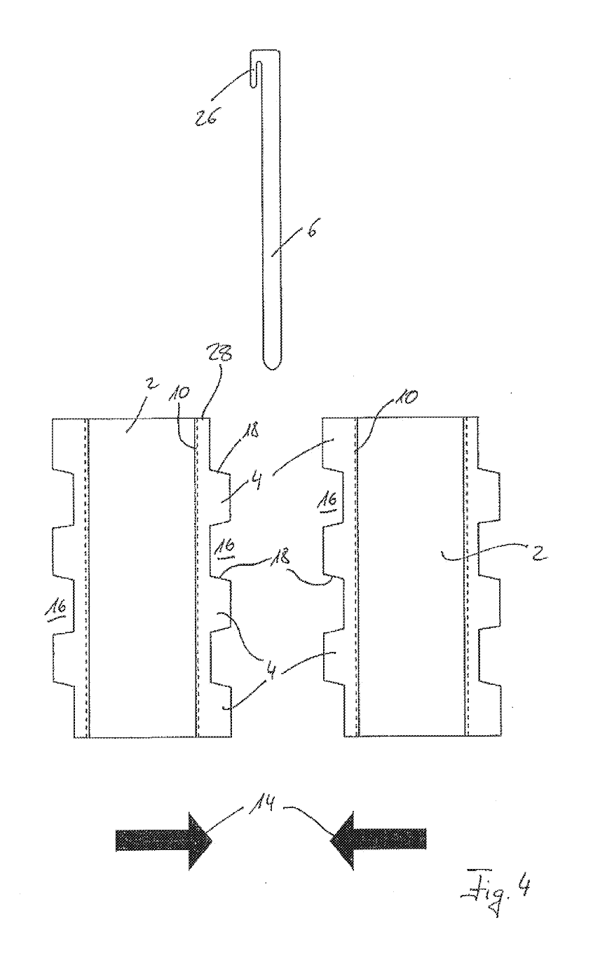

FIGS. 4 to 6 show the illustrations from FIGS. 1 to 3 for two textile segments of a bandage according to a second exemplary embodiment of the present invention; and

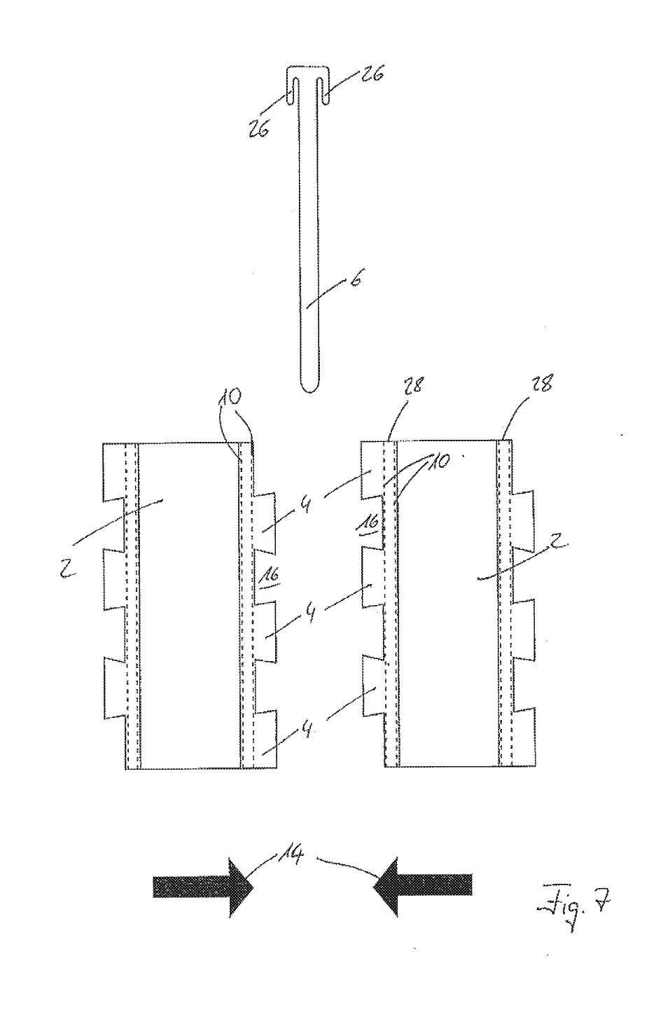

FIGS. 7 to 9 show the illustrations from FIGS. 1 to 3 for two textile segments for a bandage according to a third exemplary embodiment of the present invention.

FIG. 1 shows two textile segments 2 for a bandage (not shown in more detail) according to a first exemplary embodiment of the present invention. The textile segments 2 on the left and right peripheries thereof in FIG. 1 dispose of loops 4 which in the exemplary embodiment shown can be disposed such that said loops form an almost or a completely continuous tunnel through which a connecting bar 6 can he fitted by sliding the latter therethrough. In the exemplary embodiment shown, the loops 4 are configured so as to be integral to a segment body 8 and are formed by folding over or folding back a peripheral region which subsequently is connected to the segment body 8 by way of a seam 10 such that the loops 4 are configured. The segment body 8 in the exemplary embodiment shown, like the loops 4, is composed of a textile material.

The textile segments 2 according to FIG. 1 moreover have flaps 12 which in the exemplary embodiment shown are fastened, for example adhesively bonded or sewn, to the segment body 8.

In order for the, two textile segments 2 to be connected, the latter are displaced toward one another along the arrows 14 until the respective loops 4 are fitted by sliding into intermediate spaces 16 that are provided to this end on the respective other textile segment 2. The lengths from top to bottom of the loops 4 in FIG. 1 advantageously correspond to the lengths of the respective intermediate spaces such that end laces 18 of the loops 4 of neighboring textile segments 2 can bear on one another. On account thereof, a complete continuous tunnel into which the connecting bar 6 can be introduced is formed. This situation is illustrated in FIG. 2. The two textile segments 2 which have now been displaced toward one another so far that the loops 4 form a continuous tube or tunnel can be seen herein, wherein the end faces 18 of loops 4 which now bear on one another in a neighboring manner bear on one another. The connecting bar 6 in this state can be displaced along the arrow 20 such that said connecting bar 6 in the exemplary embodiment shown is completely covered by the loops 4 and completely surrounded by the latter. Releasing the two textile segments 2 without first removing the connecting bar 6 is thus no longer possible. The loops 12 in the position shown in FIG. 2 are disposed on the upper and the lower end of the tunnel that is formed. by the loops 4.

FIG. 3 shows the situation after the connection bar 6 has been fitted by sliding in, said connection bar 6 therefore no longer being identifiable. The textile segments 2 are interconnected, and the flaps 12 that in FIG. 3 are central are closed by a folding over movement which is indicated by the arrows 22. A hook-and-pile fastener element which interacts with a corresponding counter element which is disposed on the segment body 8 or on the respective loop 4, for example, can be disposed herein on an inside 24 of the flaps 12. In this way, the folded-over flaps 12 can be fastened in a simple manner such that inadvertent opening and thus inadvertent slipping out of the connecting bar 6 can be impeded or completely avoided.

Two textile segments 2 for a bandage according to a second exemplary embodiment of the present invention. Said two textile segments 2 also dispose of loops 4 which now, however, as opposed. to those in FIGS. 1 to 3, do not form any intermediate spaces 16 that are perpendicularly delimited. In particular, the end faces 18 are configured so as not to be perpendicular to the longitudinal extent of the individual textile segments 2. On account thereof, the individual textile segments 2 can be more easily joined together prior to the connecting bar 6 being introduced into the loops 4. The textile segments 2 in FIG. 4 are also joined together along the arrows 14.

A hook element 26 which will engage in a cavity provided therefor when the connecting bar 6 is fitted by sliding sufficiently far into the loops 4 is located at the upper end of the connecting bar 6. It can be seen that the loops 4 are formed by a seam 10, wherein an introduction opening 28 is formed at the upper side in the textile segment 2 on the left in the figure. The hook element 26 can engage in said introduction opening 28 when the connecting bar 6 in FIG. 4 is fitted by sliding downward into the loops 4. It can be seen that the individual textile segments 2 in the exemplary embodiment shown in FIG. 4 do not have any flaps 12 for closing the loops.

It has been proven particularly advantageous for a loop 4 that is located on the periphery of the textile segment 2, for example the lowermost loop 4 in FIG. 4, to be closed at the lower end, that is to say on the external periphery of the loop 4. This can be established by a seam, for example. In the exemplary embodiment shown, the length of the connecting bar 6 corresponds to the length of the textile segments 2 in the connection direction, thus from top to bottom in FIG. 4. In this way, an ideal “erection” of the textile segments 2 can be achieved.

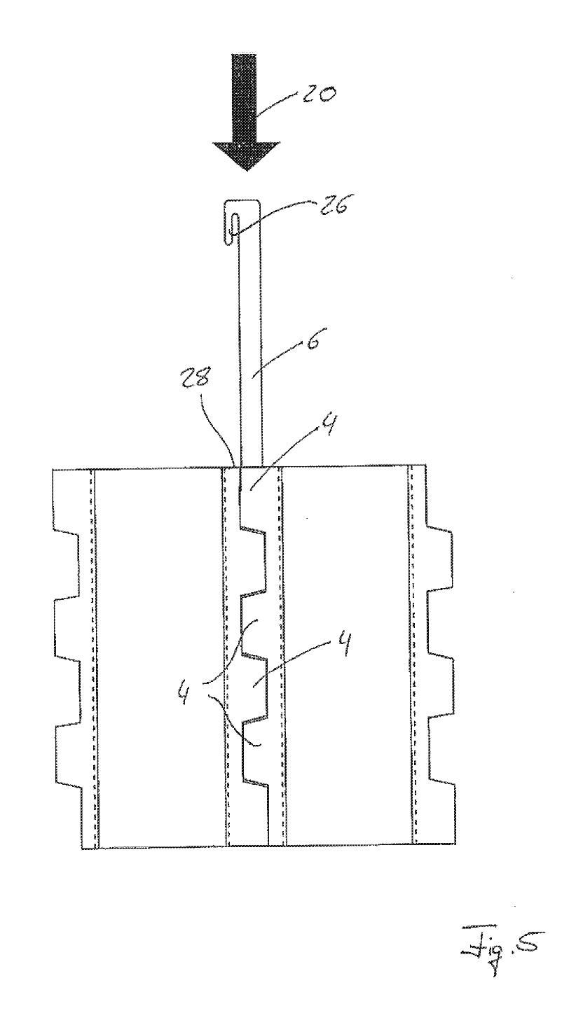

FIG. 5 shows the situation from FIG. 4 in which the connecting bar 6 is fitted by sliding into the loops along the arrow 20. The hook element 26 herein is moved toward the introduction opening 28.

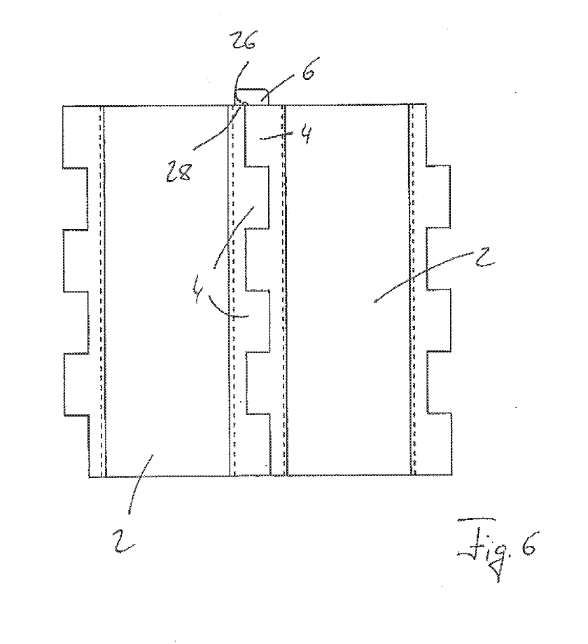

The two textile segments 2 which are interconnected are shown in FIG. 6. The connecting bar 6 has been fitted by sliding into the loops 4 and the hook element 26 has been. introduced into the introduction opening 28 that is provided for the latter. It is prevented on account thereof that the connecting bar 6 in FIG. 6 slips downward through the loops 4.

FIG. 7 shows the situation from FIG. 1 for two textile segments 2 for a bandage according to a third exemplary embodiment of the present invention. The loops 4 are configured in a dove-tail shaped manner, on account of which the intermediate spaces 16 also have a corresponding shape. Here too, the two textile segments 2 are moved toward one another in the direction of the arrows 14 until the connecting bar 6 can be fitted by sliding into the loops 4. On account of the special shape of the loops it can be expedient or necessary for the textile segments to be moved also or only in a direction that is perpendicular to that direction that is indicated by the arrows 14. Two hook elements 26 are located at the upper end of the connecting bar 6.

In each case two seams 10 are present on each side on the textile segments 2, on account of which introduction openings 28 are created on the upper and lower sides. The two hook elements 26 can engage in these introduction openings 28. Of course, it is also possible for dove-tail shaped loops 4 to be used without the connecting bar 6 having to dispose of hook elements 26. The reverse case, that is to say the use of hook elements 26 on the connecting bar 6 without the use of dove-tail shaped loops 4, is also possible, of course.

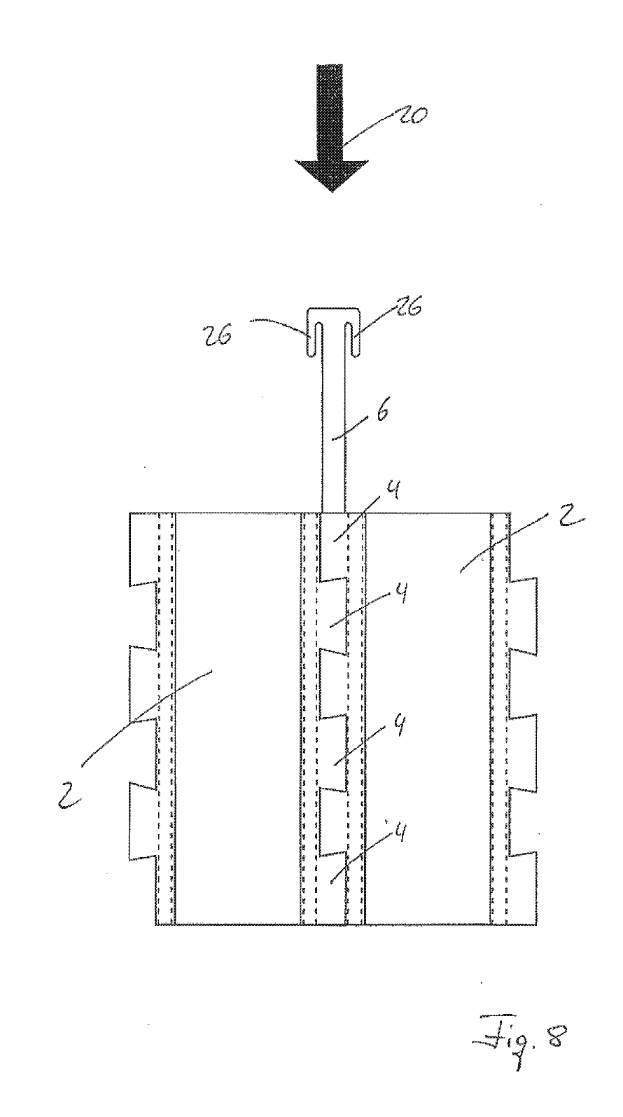

The situation from FIG. 2 for the textile elements 2 from FIG. 7 is shown in FIG. 8. The connecting bar 6 is fitted by sliding into the loops 4 along the direction of the arrow 20. The introduction of the connecting bar 6 is simplified on account of the dove-tail shaped design of the loops 4 since the two textile segments 2 in FIG. 8 can be displaced to the right and left and thus be mutually separated only with difficulty.

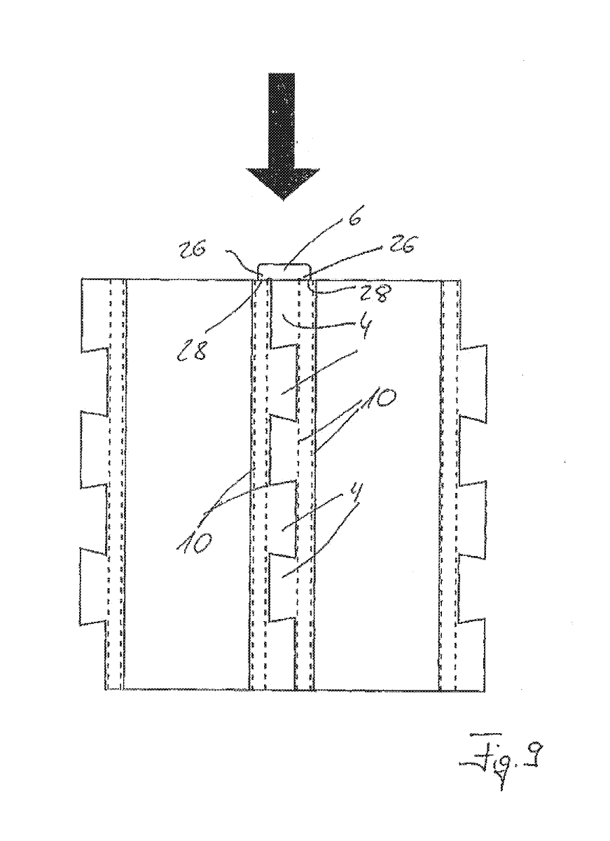

FIG. 9 shows the situation from FIG. 8 once the connecting bar 6 has been completely fitted by sliding into the loops 4. The two hook elements 26 have been introduced into the introduction openings 28 between the in each case two seams 10.

### LIST OF REFERENCE SIGNS

2 Textile segment
4 Loop
6 Connecting bar
8 Segment body
10 Seam
12 Flap
14 Arrow
16 Intermediate space
18 End face
20 Arrow
22 Arrow
24 Inside
26 Hook element
28 Introduction opening

1. A bandage comprising:
at least two textile segments which are interconnectable, wherein each of the textile segments has at least one loop of a textile material;
at least one connecting bar slidable through the at least one loop of one of the textile segments and the at least one loop of another one of the textile segments.

2. The bandage as claimed in claim 1, wherein the textile segments are composed mainly of a textile material, and the at least one loop of the textile segments is composed at least partially of the textile material.

3. The bandage as claimed in claim 2, wherein the at least one loop is configured in an integral manner from that part of the textile segments that is composed of the textile material.

4. The bandage as claimed in claim 1, wherein the at least one loop of textile segments is reinforced with a reinforcement element which is disposed on an inside of the at least one loop.

5. The bandage as claimed in claim 4, wherein the reinforcement element is composed of a plastics material and is configured as a tubular or a tube-sock element.

6. The bandage as claimed in claim 4, wherein the reinforcement element is adhesively bonded into the at least one loop.

7. The bandage as claimed in claim 1, wherein at least one of the textile segments has at least two loops.

8. The bandage as claimed in claim 1, wherein the at least one loop of textile segments is closable by at least one closure element.

9. The bandage as claimed in claim 8, wherein the at least one closure element is a foldable flap which in a closed position is fastenable by a hook-and loop fastener.

10. The bandage as claimed in claim 1, wherein the at least one connecting bar has a non-circular cross section having an oval or rectangular shape.

11. The bandage as claimed in claim 1, wherein the at least one connecting rod has one or more of at least one fastening element and at least one hook element.

12. A bandage, comprising:
first and second textile segments, the textile segments being interconnectable, each of the textile segments having at least one loop of a textile material;
at least one connecting bar slidable through the at least one loop of the first textile segment and the at least one loop of the second textile segment.

13. The bandage as claimed in claim 12, wherein the textile segments are composed mainly of a textile material, and the at least one loop of the textile segments is composed at least partially of the textile material.

14. The bandage as claimed in claim 13, wherein the at least one loop is configured in an integral manner from the part of the textile segment that is composed of the textile material.

15. The bandage as claimed in claim 12, wherein the at least one loop of the textile segments is reinforced with a reinforcement element which is disposed on an inside of the at least one loop.

16. The bandage as claimed in claim 15, wherein the reinforcement element is composed of a plastic material and is configured as a tubular or a tube-sock element.

17. The bandage as claimed in claim 15, wherein the reinforcement element is adhesively bonded into the at least one loop.

18. The bandage as claimed in claim 12, wherein at least one of the textile segments has at least two loops.

19. The bandage as claimed in claim 12, wherein the at least one loop of textile segments is closable by at least one closure element.

20. The bandage as claimed in claim 19, wherein the at least one closure element is a foldable flap, which in a closed position is fastenable by a hook-and-loop fastener.
